(19) United States
(12) Patent Application Publication
(10) Pub. No.: US 2026/0109762 A1
(43) Pub. Date: Apr. 23, 2026

(54) METHODS FOR PRODUCING ANTIBODIES

(71) Applicant: NEOMAB BIOTECHNOLOGY CO., LTD, Suzhou (CN)

(72) Inventors: Mingkun ZHANG, Suzhou (CN); Junchao PENG, Suzhou (CN); Song LI, Suzhou (CN); Cunxiang JU, Suzhou (CN); Xiang GAO, Suzhou (CN); Jing ZHAO, Suzhou (CN)

(73) Assignee: NEOMAB BIOTECHNOLOGY CO., LTD, Suzhou (CN)

(21) Appl. No.: 19/420,692

(22) Filed: Dec. 15, 2025

Related U.S. Application Data
(63) Continuation-in-part of application No. PCT/CN2024/084147, filed on Mar. 27, 2024.

(30) Foreign Application Priority Data
Oct. 25, 2023 (CN) 202311388558.7

(51) Int. Cl. C07K 16/28; A01K 67/0276; C12N 5/0775

(52) U.S. Cl. CPC C07K 16/28; A01K 67/0276; C07K 16/2803; C12N 5/0663; A01K 2217/075; A01K 2227/105; C07K 2317/14; C07K 2317/21

(57) ABSTRACT

Provided is a method for producing an antibody and the antibody produced by the method. The method includes immunizing an animal transplanted with stems cell with differentiation potential using an antigen and obtaining an antigen-specific antibody. The stem cells with differentiation potential are derived from a donor animal carrying one or more human immunoglobulin variable region gene segments. The method solves problems in the prior art including long transportation time and high costs associated with live transgenic animals used for producing human antibodies due to policy and quarantine requirements. When producing antibody against a target antigen having high homology with a transgenic animal, an immunosuppressed animal with a target antigen-encoding gene knockout is used as a transplantation recipient, which solves the problem in the prior art where transgenic animals have difficulty generating antibodies against homologous regions of the target antigen between humans and transgenic animals.

### CROSS-REFERENCE TO RELATED APPLICATIONS

This application is a continuation-in-part application of International Application No. PCT/CN2024/084147, filed on Mar. 27, 2024, which claims priority to Chinese Patent Application No. 202311388558.7, filed on Oct. 25, 2023, the entire contents of each of which are hereby incorporated by reference.

### TECHNICAL FIELD

The present disclosure generally relates to a field of biomedicine, and in particular to a method for producing a human antibody.

### SEQUENCE LISTING

This application contains a Sequence Listing which has been submitted electronically in XML format and is hereby incorporated by reference in its entirety. The XML copy, created on Dec. 8, 2025, is named “2025 Dec. 8-SequenceListing-20948-0003US00” and is 11,010 bytes in size.

### BACKGROUND

Antibody drugs are currently rapidly increasing in number and most advanced and effective class of drugs. The advent of monoclonal antibody technology has made the research and production of the antibody drugs a reality. After more than thirty years of development, therapeutic monoclonal antibody drugs have become one of the most important components of biopharmaceuticals, have broad application prospects in disease treatment, and have been successfully used to treat various diseases including tumors, autoimmune diseases, infectious diseases, and transplant rejection. Since the first therapeutic monoclonal antibody was approved in 1975, more than 100 monoclonal antibodies have been approved for marketing, showing a rapid growth trend in recent years.

The earliest therapeutic antibodies were completely animal-derived. Their immunogenicity causes the human body to produce human anti-mouse antibodies (HAMA) and even leads to serious side effects such as allergies. Humanization of animal-derived antibodies through antibody engineering can reduce immunogenicity. However, this process requires enormous cost and time, and it is difficult to completely eliminate immunogenicity while maintaining high affinity. The solution is to develop fully human antibodies. Currently, there are four main manners: A) display technology, B) immunization screening of transgenic mice, C) immortalization of human B cells, and D) single-cell screening of human memory B cells. Among them, the “display technology” can screen antibodies with a high throughput and a high speed. However, because the screening process is purely artificial in vitro system, the obtained antibodies have limited diversity and affinity, contain non-natural structures, and still have immunogenicity, requiring a lot of time and cost for optimization and modification. The human B cells can only be isolated from humans who have been exposed to an antigen and have an immune response. Due to source limitations, screening manners using the human B cells can obtain only a few types of antibodies, and the probability of screening high-affinity antibodies is very low. Compared with other manners, genetic modification mouse models carrying human antibody-encoding genes have obvious advantages. A mouse carrying complete human antibody variable region encoding genes (>3 Mb) can produce immune responses close to those of humans against antigens, and the fully human antibody with high-affinity can be obtained by human genes encoding and natural maturation processes in vivo such as somatic hypermutation. Because the fully human antibody relies on the completely natural B cell screening and maturation process, no post-optimization is required. The antibodies are encoded by human genes, so there is no immunogenicity problem. This manner can obtain high-affinity monoclonal antibodies with far less time and cost than other manners. For example, the HuMab platform developed by Medarex, the Trianni Mouse developed by Trianni, the VelocImmune developed by Regeneron, and the Kymouse developed by Kymab all belong to this type of model.

The mouse carrying human antibody-encoding genes can have a sound immune system. Immunization with a target antigen can produce high-affinity antibodies encoded by human genes, which greatly simplifies the technical process of therapeutic antibody discovery and saves the time and cost of antibody engineering (e.g., humanization and affinity improvement).

However, there is high homology between mouse proteins and human proteins. Taking CLAUDIN-18 as an example, the amino acid homology of CLAUDIN-18 between a human and the mouse is 88%. The homology of some proteins between a human and the mouse can reach more than 95%. The B cell maturation process includes a “negative selection” process, which eliminates B cell clones that recognize the body's own proteins. The negative selection mechanism makes it difficult for the mouse to produce antibodies against the human-mouse homologous regions of proteins, becoming a major obstacle to obtaining antibodies against human targets. To overcome this difficulty, in the prior art, a gene encoding a target antigen in a mouse carrying a human antibody-encoding gene is knocked out, and a transgenic mouse with a knockout homozygote for immunization is obtained. Although the manner can obtain antibodies against homologous antigens (sequence-conserved regions), it requires creating a gene knockout strain for each target antigen, which consumes a lot of time. In addition, when it is necessary to produce antibodies against targets (the target antigens) related to immune system function, knocking out the gene encoding the target antigen in the mouse may affect the development and function of the mouse immune system, thereby preventing the acquisition of corresponding antibodies through immune responses.

In addition, since the immunization process is completed in the transgenic (or transgenic and knockout) mouse, live animals need to be obtained to conduct experiments. Limited by policies, quarantine, and other related requirements, the transportation of the live animals is time-consuming and expensive, which also reduces the accessibility of transgenic live animals and brings difficulties to using the transgenic mice for antibody screening.

### SUMMARY

One or more embodiments of the present disclosure provide a method for producing an antibody. The method comprises immunizing a recipient animal with an antigen and obtaining an antigen-specific antibody, wherein the recipient animal is an animal transplanted with stem cells with differentiation potential by immune system reconstitution, the stem cells with differentiation potential are derived from a donor animal carrying one or more human immunoglobulin variable region gene segments, and the recipient animal is a non-human mammal.

In some embodiments, the method includes the following steps.
(a) subjecting the recipient animal to immunosuppressive pretreatment to obtain an immunosuppressed recipient animal; providing the donor animal carrying one or more human immunoglobulin variable region gene segments, and collecting the stem cells with differentiation potential from the donor animal and transplanting the stem cells into the immunosuppressed recipient animal to obtain an immune-reconstituted recipient animal with immune cells derived from the donor animal.
(b) immunizing the immune-reconstituted recipient animal obtained in step (a) with the antigen.
(c) confirming that the immunized immune-reconstituted recipient animal is an animal capable of producing an antibody binding to the antigen, collecting immune cells of the recipient animal, and further producing a human antibody by using the collected immune cells.

One or more embodiments of the present disclosure provide a human antibody, which is produced by the method above.

One or more embodiments of the present disclosure provide a pharmaceutical composition. The pharmaceutical composition includes the human antibody above.

One or more embodiments of the present disclosure provide an immune-reconstituted mouse, which is transplanted with stem cells with differentiation potential, and the stem cells with differentiation potential are derived from a transgenic mouse carrying one or more human immunoglobulin variable region gene segments.

### BRIEF DESCRIPTION OF THE DRAWINGS

FIG. 1 is a diagram showing body weight changes of B6 recipient mice after transplantation with bone marrow from an EGFP transgenic mouse according to some embodiments of the present disclosure.

FIG. 2 is a diagram showing survival rate of the B6 recipient mice after transplantation with the bone marrow from the EGFP transgenic mouse according to some embodiments of the present disclosure.

FIG. 3 is a diagram showing results of immune cell origin analysis for the B6 recipient mice after transplantation with the bone marrow from the EGFP transgenic mouse according to some embodiments of the present disclosure.

FIG. 4 is a diagram showing body weight changes of genetically modified recipient mice and B6 wild-type recipient mice after transplantation with bone marrow cells from a transgenic mouse carrying human immunoglobulin variable region encoding genes according to some embodiments of the present disclosure.

FIG. 5 is a diagram showing survival rate of the genetically modified recipient mice and the B6 wild-type recipient mice after transplantation with the bone marrow cells from the transgenic mouse carrying the human immunoglobulin variable region encoding genes according to some embodiments of the present disclosure.

FIG. 6 is a diagram showing results of immune serum antibody titer determination for the genetically modified recipient mice and the B6 wild-type recipient mice after transplantation with the bone marrow cells from the transgenic mouse carrying the human immunoglobulin variable region encoding genes according to some embodiments of the present disclosure.

FIG. 7 is a diagram showing binding activity of anti-PD1 antibodies, screened from PD1 KO recipient mice transplanted with the bone marrow cells from the transgenic mouse carrying the human immunoglobulin variable region encoding genes, to recombinant hPD1 proteins according to some embodiments of the present disclosure.

FIG. 8 is a diagram showing binding activity of the anti-PD1 antibodies, screened from the PD1 KO recipient mice transplanted with the bone marrow cells from the transgenic mouse carrying the human immunoglobulin variable region encoding genes, to Jurkat-PD1 cells according to some embodiments of the present disclosure.

FIG. 9 is a diagram showing in vivo efficacy results of the anti-PD1 antibodies (9G4C5 and 6F2A11) screened from the PD1 KO recipient mice transplanted with the bone marrow cells from the transgenic mouse carrying the human immunoglobulin variable region encoding genes according to some embodiments of the present disclosure.

FIG. 10 is a diagram showing body weight changes of mice reconstituted by fetal liver cells transplantation according to some embodiments of the present disclosure.

FIG. 11 is a diagram showing survival rate of the mice reconstituted by the fetal liver cells transplantation according to some embodiments of the present disclosure.

FIG. 12 is a diagram showing results of immune cell origin analysis for the mice reconstituted by the fetal liver cells transplantation according to some embodiments of the present disclosure.

FIG. 13 is a diagram showing results of B cell origin analysis after reconstitution by transplantation of cryopreserved and thawed bone marrow cells and freshly isolated bone marrow cells according to some embodiments of the present disclosure.

FIG. 14 is a diagram showing results of immune cell origin analysis after bone marrow transplantation in TIGIT KO mice and LAG3 KO mice according to some embodiments of the present disclosure.

FIG. 15 is a diagram showing results of immune serum titer after the bone marrow (BM) transplantation in the TIGIT KO mice and the LAG3 KO mice according to some embodiments of the present disclosure.

FIG. 16 is a diagram showing results of immune cell origin analysis for the genetically modified recipient mice (TG), CLDN KO mice, and CLDN KO transplanted mice according to some embodiments of the present disclosure.

FIG. 17 is a diagram showing results of immune serum antibody titer determination for the genetically modified recipient mice (TG), the CLDN KO mice, and CLDN KO recipient mice after transplantation with the bone marrow cells from the transgenic mouse carrying the human immunoglobulin variable region encoding genes according to some embodiments of the present disclosure.

### DETAILED DESCRIPTION

The present disclosure provides a new method for producing a human antibody. The method includes transplanting stem cells from a donor transgenic animal carrying one or more human immunoglobulin variable region gene segments into an immunosuppressed recipient animal to reconstitute an immune system of the recipient animal. After successful immune system reconstitution, the recipient animal is immunized with an antigen, and immune cells derived from the donor then produce antibodies. The method addresses problems in existing technologies where production of human antibodies requires live transgenic animals that are subject to policy and quarantine requirements, resulting in long transportation times and high costs. The donor-derived immune cells can be transplanted freshly or after cryopreservation and thawing and are suitable for long-distance transportation. Cryopreserved donor immune cells (instead of live animals) can be thawed and transplanted into recipient mice to reconstitute an immune system, and antibodies are produced by immunization, thereby avoiding difficulties associated with transporting the live animals.

When producing antibodies against a target antigen having high homology between the transgenic animal and human, the method in the present disclosure can use an immunosuppressed animal with a target antigen-encoding gene knockout as a transplantation recipient, which addresses the problem in existing technologies where the transgenic animals have difficulty producing antibodies against homologous regions of the target antigen between human and the transgenic animal.

Specific technical solutions of the present disclosure are as follows.

A method for producing an antibody comprises immunizing a recipient animal with an antigen and obtaining an antigen-specific antibody. The recipient animal is an animal transplanted with stem cells with differentiation potential by immune system reconstitution. The stem cells with differentiation potential are derived from a donor animal carrying one or more human immunoglobulin variable region gene segments.

In some embodiments, the human immunoglobulin comprises at least one of IgG, IgM, IgA, IgD, or IgE. The variable region is at least one of regions encoded by gene segments formed by a human V_{H}D_{H}J_{H} rearrangement, a human V_{K}J_{K} rearrangement, or a human V_{L}J_{L} rearrangement. In some embodiments, the variable region encoding segment is formed by recombination involving one of J_{H1}, J_{H1P}, J_{H2}, J_{H2P}, J_{H3}, J_{H3P}, J_{H4}, J_{H5}, or J_{H6}. In some embodiments, the variable region encoding segment is formed by recombination involving one of J_{K1}, J_{K2}, J_{K3}, J_{K4}, or J_{K5}.

In the present disclosure, the antibody is a human antibody capable of specifically recognizing and binding a target antigen.

In some embodiments, the target antigen is a molecule or a compound that is artificially synthesized, recombinantly expressed, or naturally occurring in nature.

In some embodiments, the target antigen is a polypeptide chain including more than three amino acids.

In some embodiments, the target antigen is a membrane receptor or a free protein.

In some embodiments, the human antibody is a diagnostic, detective, or therapeutic antibody targeting a specific disease-related therapeutic target. In some embodiments, the human antibody is a therapeutic antibody.

In some embodiments, the human antibody is an antibody with a variable region encoded by human genes. In some embodiments, the human antibody is a therapeutic antibody for treating human diseases such as tumors, autoimmune diseases, metabolic diseases, or neurological diseases.

In the above method, a transplanted donor and the recipient animal have the same genetic background or different genetic backgrounds.

The stem cells with differentiation potential refers to stem cells capable of differentiating into a plurality of immune cells. In some embodiments, the stem cells with differentiation potential include at least one of bone marrow cells, fetal liver cells, hematopoietic stem cells, pluripotent stem cells, or induced pluripotent stem cells (iPSCs). In some embodiments, the stem cells with differentiation potential are the bone marrow cells or the hematopoietic stem cells. In some embodiments, the stem cells with differentiation potential are freshly isolated cells or cryopreserved and thawed cells.

In the above method, the transplanted donor and the recipient animal are non-human mammals or rodents. In some embodiments, the transplanted donor and the recipient animal is selected from a group consisting of a rat, a mouse, a rabbit, a sheep, and a non-human primate. In one example of the present disclosure, the transplanted donor and the recipient animal are mice.

In the method of the present disclosure, the immunosuppressed recipient animal is obtained by one or more of myeloablation, immunosuppression and immunomodulation using a low-toxicity drug, or genetic modification to cause immune system deficiency or suppression in the recipient.

In some embodiments, the myeloablation includes eliminating hematopoietic stem cells in a bone marrow of the recipient animal using a sub-lethal dose of ionizing radiation or chemical means. The use of the low-toxicity drug refers to using a drug that inhibits the stem cells proliferation or a differentiation signaling blocker (e.g., imatinib) for the immunosuppression and the immunomodulation. The induced immunodeficiency or suppression in the immune system of the recipient animal through genetic modification includes developmental or differentiation defects of the hematopoietic stem cells, including but not limited to a c-kit or thrombopoietin gene mutation mouse.

The manner for eliminating the hematopoietic stem cells in the bone marrow of the animal using the ionizing radiation or the chemical means in the present disclosure may adopt types, doses, and operating manners conventionally used in existing art. For example, the ionizing radiation is X-ray irradiation or Co-60 gamma-ray irradiation with a radiation dose of 600 cGy-950 cGy, or a higher sub-lethal dose, specifically referring to the literature (Eunbee Park, et al., J Vis Exp, 2021). The chemical means is administering at least one of busulfan, cyclophosphamide, or melphalan to the recipient animal. Taking the busulfan as an example, two consecutive intraperitoneal injections at a dose of 20 mg/kg with a 24-hour interval can complete myeloablative pretreatment (Encarnacion Montecino-Rodriguez, et al., STAR Protocols, 2020).

The recipient animal of the present disclosure is a wild-type animal or a genetically engineered animal. The genetically engineered animal is a transgenic animal carrying the human genes, an animal carrying mutant genes, an animal expressing reporter genes, or an animal lacking target antigen-encoding genes (e.g., a target antigen-encoding gene knockout). In some embodiments, the recipient animal is the animal lacking the target antigen-encoding genes.

The manner for screening the human antibody is a hybridoma screening, a single B cell screening, display library screening, etc. A person skilled in the art understands that a B cell collection obtained by immunizing an animal obtained based on the transplantation reconstitution method provided in the present disclosure can be used to obtain a monoclonal cell line producing a target antibody through existing antibody screening technologies and further produce a target human antibody meeting requirement.

The method of the present disclosure specifically includes the following steps.
(a) subjecting the recipient animal to immunosuppressive pretreatment to obtain an immunosuppressed recipient animal, providing the donor animal carrying one or more human immunoglobulin variable region gene segments, collecting bone marrow cells, embryonic cells, or purified hematopoietic stem cells from the donor animal, and transplanting the bone marrow cells, the embryonic cells, or the purified hematopoietic stem cells into the immunosuppressed recipient animal to obtain an immune-reconstituted recipient animal with immune cells derived from the donor animal. In some embodiments, a manner for the providing the donor animal carrying the one or more human immunoglobulin variable region gene segments includes constructing or purchasing the donor animal carrying the one or more human immunoglobulin variable region gene segments.

In some embodiments, flow cytometry, immunofluorescence, quantitative polymerase chain reaction (PCR), or other corresponding technologies are used to detect a reconstruction level of immune cells in the recipient animal after transplantation. When donor-derived immune cells are stably reconstituted in the recipient, the animal may be immunized with the antigen.
(b) immunizing the immune-reconstituted recipient animal obtained in the step (a) with the antigen.
(c) confirming that the immunized immune-reconstituted recipient animal is an animal capable of producing an antibody binding to the antigen, collecting immune cells from the recipient animal, and further producing a human antibody by using the collected immune cells.

In some embodiments, serum from the immunized animal is collected. An enzyme-linked immunosorbent assay (ELISA) or other similar technology is used to detect a serum antibody titer capable of binding to the target antigen. The animal capable of producing the antibody binding to the target antigen is selected. Immune cells are collected from animals. The hybridoma screening, flow sorting, high-throughput sequencing, etc., are used to screen clones capable of producing monoclonal antibodies with parameters such as affinity and functional activity meeting requirements. An antibody encoding region is sequenced to obtain an encoding sequence. The antibody variable region sequence is further combined with the human antibody constant region. The antibody is produced by methods such as recombinant expression.

In the above method, the order of constructing the transplanted donor and the recipient animal is not limited.

Some embodiments of the present disclosure provide a human antibody produced by the method of the present disclosure. Some embodiments of the present disclosure provide a pharmaceutical composition including the human antibody produced by the method of the present disclosure.

The present disclosure further provides an immune-reconstituted mouse. The immune-reconstituted mouse is transplanted with stem cells with differentiation potential. The stem cells with differentiation potential are derived from a transgenic mouse carrying one or more human immunoglobulin variable region gene segments.

In some embodiments, the stem cells with differentiation potential include at least one of the bone marrow cells, the fetal liver cells, the hematopoietic stem cells, the pluripotent stem cells, or the induced pluripotent stem cells (iPSCs).

In some embodiments, the mouse is a wide-type mouse or a target antigen-encoding gene knockout mouse, which is capable of producing the human antibody against the target antigen after antigen immunization.

In some embodiments, the immune-reconstituted mouse is obtained by inducing immunodeficiency or suppression in an immune system of a mouse by at least one of myeloablation, immunosuppression and immunomodulation using a low-toxicity drug, and genetic modification; and transplanting the stem cells with differentiation potential into the mouse.

In some embodiments, the human immunoglobulin is selected from a group consisting of IgG, IgM, IgA, IgD, and IgE.

In some embodiments, the variable region is at least one of regions encoded by gene segments formed by human V_{H}D_{H}J_{H} rearrangement, human V_{K}J_{K} rearrangement, or human V_{L}J_{L} rearrangement.

The present disclosure has the following advantages.
(1) Due to policy and quarantine requirements, transportation of the live animals used for producing an antibody in existing art is time-consuming and expensive. The method of the present disclosure allows pre-obtained donor-derived immune cells to be cryopreserved, which is easily transported over long distances. The thawed donor-derived immune cells (instead of the live animals) can be transplanted into local or remote recipient mice to reconstitute an immune system. The mice are then immunized with the target antigen to produce antibodies, avoiding difficulties in transporting the live animals.
(2) In the existing art, when the transgenic mouse is used to produce the human antibody, the transgenic mouse carrying the human antibody-encoding genes are directly immunized, which requires a large number of such humanized transgenic animals, which are often expensive. The transplantation and reconstitution method in the present disclosure can multiply save the usage of the transgenic animals. Stem cells from a single donor mouse can reconstitute three or more recipient mice to obtain more animals for immunization, which reduces costs.
(3) In the existing art, the gene encoding the target antigen in the transgenic mouse carrying the human antibody-encoding gene is knocked out to establish a genetically modified strain that cannot express the target antigen, which is then used for antibody screening against a specific target. However, this manner requires the production of a gene knockout strain for each target antigen, which is time-consuming and costly. To overcome the problem that immunocompetent animals cannot generate antibodies against human-animal homologous antigens, the transgenic animal carrying the human antibody-encoding gene (the transplantation donor) and the transgenic animal with the target antigen-encoding gene knockout (the transplantation recipient) are constructed in the present disclosure. Through stem cell transplantation, a functionally normal immune cell population derived from the donor animal is reconstituted in the transgenic animal with the target antigen-encoding gene knockout (the transplantation recipient). The functionally normal immune cell population includes mature functional T cells, B cells, antigen-presenting cells, and other immune cells with complete functions. The stem cells with differentiation potential from the donor differentiate and mature in the transgenic animal with the target antigen-encoding gene knockout (the recipient). The T cells and the B cells undergo the negative selection in the transplantation recipient. Since the target antigen is absent in the recipient after transplantation and reconstitution, the “negative selection” against the target antigen cannot effectively eliminate the B cell clones that produce antibodies recognizing the human/animal homologous region of the target antigen. Thus, the transgenic animal with the target antigen-encoding gene knockout that has undergone successful immune reconstitution can utilize the immune cells of the antibody-encoding gene humanized animal to produce antibodies encoded by human genes.
(4) The problem that ordinary animals (the mice) have difficulty obtaining antibodies against homologous antigens (such as conserved regions of amino acid sequences of homologous proteins) is solved in the present disclosure. The transgenic animals (the recipients) with the antigen-encoding gene knockout are used for the development of therapeutic antibodies against corresponding targets, resulting in increased antibody repertoire diversity, enhanced immune response, and shortened immunization cycle, which is of extremely important value for screening therapeutic antibodies.
(5) The manner of the bone marrow (or the hematopoietic stem cell) transplantation is adopted in the present disclosure, which can use the bone marrows (or the hematopoietic stem cells) of the same type of transgenic ordinary animals (mice) to reconstitute the immune system in different recipient animals (the mice), achieving personalization of antibody production.
(6) The recipient animal used in the present disclosure can be a wild-type animal or a genetically modified strain. For example, the recipient animal can be a disease (e.g., autoimmune diseases, tumors, and metabolic diseases) model carrying genetic modifications (e.g., gene mutations or transgenes). The model can be used to screen antibodies against potential therapeutic or detection targets for specific diseases.

The recipient animal can also be a genetically modified animal carrying a specific reporter gene expression system. For example, when the animal produces the target antibody, specific tissues or cells can exhibit phenotypes such as fluorescence or cytokine secretion, assisting in antibody screening.
(7) The stem cell transplantation and reconstitution adopted in the present disclosure is a non-genetic method, which does not require target gene knockout on the transgenic mouse, saving the long and complex process of genetic modification and mouse breeding, thereby improving efficiency.
(8) The target antigen-encoding gene knockout animal (the recipient) used for transplantation in the present disclosure can be produced simultaneously with the transplantation donor animal or can be produced in large batches and for multiple targets. These gene knockout models themselves do not carry human antibody-encoding genes and can also be used for another research.
(9) The hematopoietic stem cells collected from the transgenic animal with the target antigen-encoding gene knockout (the recipient) in the present disclosure can be cryopreserved and transplanted after thawing. The stem cells themselves can serve as a non-living product, which is easy for batch production, preservation, and transportation, and can be thawed, transplanted, and immunized for antibody screening according to experimental requirements.

The specific steps of the present disclosure are illustrated below by examples but are not limited by the examples.

The terms used in the present disclosure, unless otherwise specified, generally have the meanings usually understood by those of ordinary skill in the art.

The present disclosure is further described in detail below with reference to specific examples and data. It should be understood that these examples are only for illustrating the present disclosure and do not limit the scope of the present disclosure in any way.

In the following examples, various processes and manners not described in detail are conventional manners well known in the art.

### Example 1 Construction of Immune-Reconstituted Mice Via Bone Marrow Cell Transplantation

### Immunosuppressive Pretreatment of Recipient Mice

4-week-old B6 mice (products of Jiangsu GemPharmatech Co., Ltd., a strain number of N000013) were selected as recipients and the irradiation myeloablation manner for pretreatment was used. However, those skilled in the art understand that animals of other ages and strains may serve as transplantation recipients, and other pretreatment manners that achieve myeloablative effects (such as chemical myeloablation) may also be used. The research suggests that 600 cGY-900 cGY is a suitable myeloablative irradiation dose for the selected recipients, so 600 cGY-900 cGY is selected as the irradiation dose. To improve the survival rate of mice after irradiation, antibiotics is optionally administered before the irradiation.

### Extraction and Transplantation of Donor Mouse Bone Marrow Cells

The bone marrow (BM) of an enhanced green fluorescent protein (EGFP) transgenic donor mouse was transplanted into the background strain C57BL/6JGpt (B6) that had undergone irradiation pretreatment. The EGFP transgenic donor mouse was a fluorescent mouse model (a strain number of T006163) developed by Jiangsu GemPharmatech Co., Ltd. The EGFP is controlled by the CAG promoter and is widely expressed in mouse tissues. The model can be used to obtain various mouse cells labeled with the green fluorescent protein and is particularly applied in cell transplantation research.

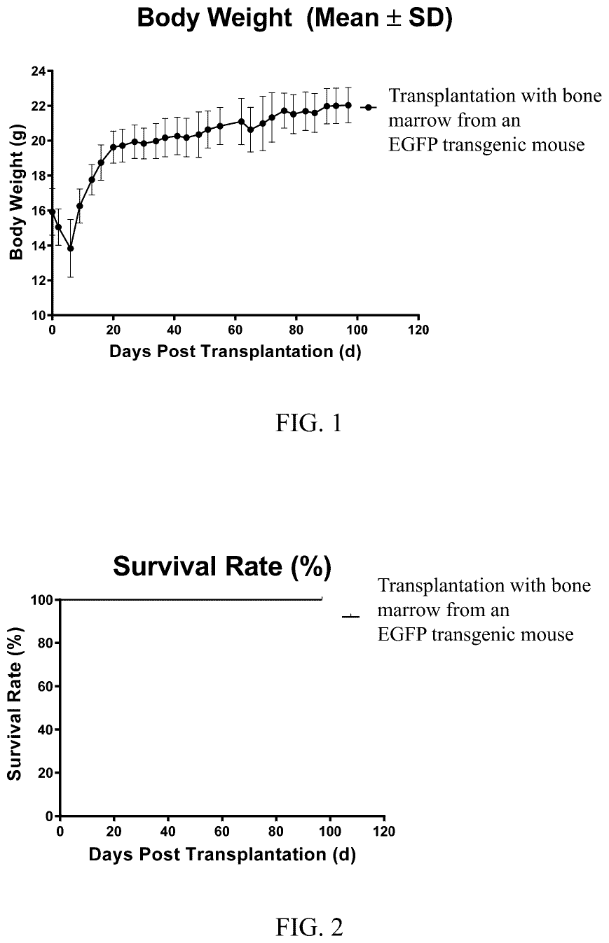

The specific operational steps were as follows. The donor mouse was euthanized, and the hind leg bones were dissected and placed in a sterile culture dish containing 4° C. PBS. PBS was used to flush the bone marrow to obtain bone marrow cells from the donor mouse. After resuspension and red blood cell lysis, the bone marrow cells were collected for later use. The collected bone marrow cells were transplanted into the irradiated recipient mice via tail vein injection. The mice were observed and weighed weekly after transplantation to record body weight and survival rate. FIG. 1 is a diagram showing body weight changes of B6 recipient mice after transplantation with bone marrow from an EGFP transgenic mouse. The results show that the body weight of the irradiated and transplanted recipient mice recovers with increasing age. The body weight change trend indicates that the physiological state of the transplanted mice is normal. FIG. 2 is a diagram showing survival rate of the B6 recipient mice after transplantation with the bone marrow from the EGFP transgenic mouse. The results show that the survival rate of the irradiated and transplanted recipient mice is 100%. No mouse death occurs during the observation period of over 90 days post transplantation, which indicates that the transplanted mice (100%) can survive for at least 90 days or more.

### Assessment of Immune System Reconstitution

In this example, the cells from the EGFP transgenic donor mouse are fluorescent, while the cells from the recipient B6 wild-type mouse are non-fluorescent. Therefore, B cells (mCD19+) in the peripheral blood of the reconstituted mouse were detected using flow cytometry. If green fluorescence is detected, it indicates that the cells are derived from the donor. If no fluorescence is detected, it indicates that the cells are derived from the recipient mouse.

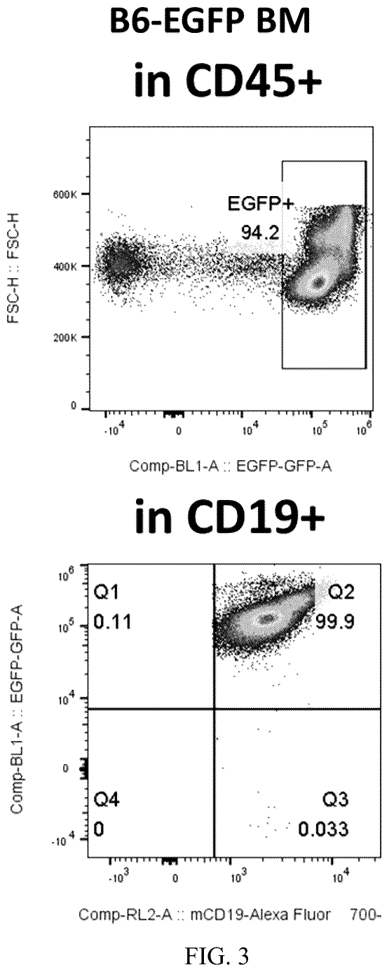

Starting from the 4th week post transplantation, the proportion of donor-derived immune cells in the peripheral blood of the reconstituted mice was assessed. Using EGFP as a fluorescent marker, the proportion of donor-derived immune cells was detected by the flow cytometry. FIG. 3 is a diagram showing results of immune cell origin analysis for the B6 recipient mice after transplantation with the bone marrow from the EGFP transgenic mouse. The results show that 94.2% of the white blood cells (mCD45+) and 99.9% of the B cells (mCD19+) in the recipient mice 60 days post irradiation and transplantation are EGFP+ cells, and thus derived from the donor, which proves that the donor-derived bone marrow cells can stably reconstitute immune cells in the recipient mouse, and the recipient's own immune cells do not reappear after immunosuppression. This example proves that the method of the present disclosure can reconstitute the immune system by transplanting bone marrow cells from the donor mouse into an immunosuppressed pretreated recipient.

### Example 2 Production of Specific Antibodies Using Bone Marrow Transplantation Immune-Reconstituted Mice

Using the transplantation and reconstitution method described in Example 1, bone marrow cell transplantation reconstituted animals were constructed according to the groups in Table 1. G1 and G3 were PD1 gene knockout recipient mice (PD1 KO) (the recipient mice were products of Jiangsu GemPharmatech Co., Ltd., a strain number of T011515) and wild-type B6 mice (the recipient mice were products of Jiangsu GemPharmatech Co., Ltd., a strain number of N000013) that were transplanted with bone marrow cells from human immunoglobulin variable region gene segment humanized mice (TG), respectively. G2 and G4 were PD1 gene knockout recipient mice (PD1 KO) and wild-type B6 mice that did not undergo immunosuppressive pretreatment and transplantation, respectively. The donor mouse used in this experiment carried human immunoglobulin heavy chain (IGH) variable region encoding genes and human immunoglobulin kappa chain (IGK) variable region encoding genes in its genome. Additionally, corresponding mouse Igh and Igk genes were inactivated (obtained by the manner disclosed in E-Chiang L et al., Nature Biotechnology. 2014). Specifically, by referring to the embryonic stem cell gene targeting manner described in the literature, targeting vectors carrying human IGH variable region gene segments (including human VHI, Du, and Ju gene segments) and targeting vectors carrying human IGK variable region gene segments (including human V_{K} and J_{K} gene segments) were constructed. The targeting vectors above were electroporated into embryonic stem (ES) cells, respectively, to obtain ES clones with successful integration of the human IGH gene segments upstream of the mouse Igh gene constant region encoding segment and ES clones with successful integration of the human IGK gene segments upstream of the mouse Igk gene constant region encoding segment. The above targeted clones were injected into mouse blastocysts and transplanted into the uteri of pseudopregnant mice, ultimately obtaining targeted mice developed from the targeted ES cells. The IGH targeted mice were mated with the IGK targeted mice to obtain homozygous IGH IGK double-targeted mice, which served as humanized mice carrying human immunoglobulin variable region encoding genes (the donors) to provide stem cells with differentiation potential for transplantation.

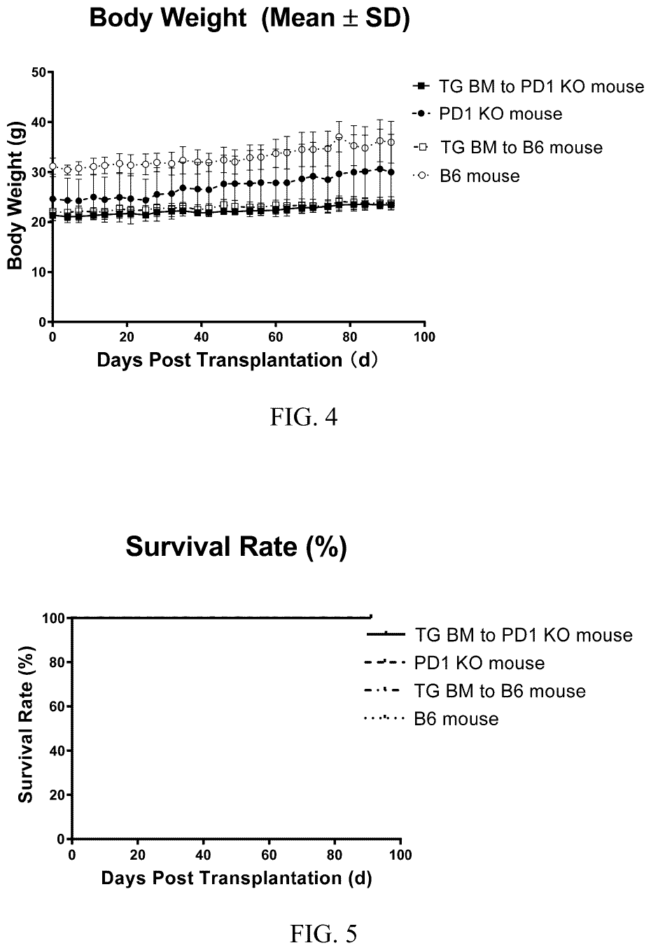

Post transplantation, mice were observed and weighed weekly to record body weight and survival rate. The results show that the recipient mice grow stably after transplantation, and 100% of the recipient mice survive for over 90 days post transplantation (shown in FIG. 4 and FIG. 5). After successful reconstitution, immunization was performed using commercially available recombinant hPD1 protein and Freund's adjuvant.

TABLE 1
Experiment groups for bone marrow cell
transplantation reconstituted animals

| | Donor | Recipient | Donor | | |
|---|---|---|---|---|---|
| Group | Mouse | Mouse | Cell | Antigen | Frequency |
| G1 | Antibody- | PD1 | Bone | hPD1 | Administered |
| | encoding | gene | marrow | protein | once every |
| | gene | knockout | cell | and | 3 weeks, a |
| | humanized | mouse | | Freund's | total of |
| | mouse (TG) | (PD1 KO) | | adjuvant | 4 doses |
| G2 | None | PD1 | None | hPD1 | Administered |
| | | gene | | protein | once every |
| | | knockout | | and | 3 weeks, a |
| | | mouse | | Freund's | total of |
| | | (PD1 KO) | | adjuvant | 4 doses |
| G3 | Antibody- | Wild- | Bone | hPD1 | Administered |
| | encoding | type | marrow | protein | once every |
| | gene | mouse | cell | and | 3 weeks, a |
| | humanized | (B6) | | Freund's | total of |
| | mouse (TG) | | | adjuvant | 4 doses |
| G4 | None | Wild- | None | hPD1 | Administered |
| | | type | | protein | once every |
| | | mouse | | and | 3 weeks, a |
| | | (B6) | | Freund's | total of |
| | | | | adjuvant | 4 doses |

### Immunization Procedure

Primary immunization: 100 μg antigen was thoroughly mixed with an equal volume of Freund's complete adjuvant using a vortex mixer, and each mouse received subcutaneous multi-point injections. The day of the primary immunization was designated as day 0.

Second immunization: On day 21, 100 μg antigen was thoroughly mixed with an equal volume of Freund's incomplete adjuvant using the vortex mixer, and subcutaneous multi-point injections were administered with the same dose, manner, and route as the primary immunization.

Third immunization: On day 42, 100 μg antigen was thoroughly mixed with an equal volume of Freund's incomplete adjuvant using the vortex mixer, and subcutaneous multi-point injections were administered with the same dose, manner, and route as the primary immunization (if further immunization is required, the scheme is consistent).

### Antibody Titer Detection

a) Two weeks post the third immunization, approximately 100 μL-200 μL of blood was collected from the mouse orbit, centrifuged at 7000 rpm for 5 min, and the upper pale-yellow serum was taken as the sample.
b) The antigen (recombinant hPD1 protein) was diluted to different concentrations (1 μg/mL, 500 ng/ml, 250 ng/ml, 125 ng/ml, and 50 ng/ml) using coating buffer, added to the ELISA plate at 100 μl/well, with 2 replicates per concentration, and coated overnight at 4° C.
c) The coating buffer was discarded, each well was washed 3 times with 200 μl PBST for 3 min each time, the ELISA plate was inverted on blotting paper to remove excess PBST, and each well was coated with 200 μL blocking buffer at 37° C. for 2 h.
d) The blocking buffer was discarded, and each well was washed 3 times with 200 μl PBST for 3 min each time. Positive sera and negative sera were subjected to two-fold serial dilution (1:200, 1:400, 1:800, and 1:1600) with the coating buffer. 100 μL/well of the diluted sera was added to an ELISA plate, with a blank control set simultaneously. The ELISA plate was incubated at 37° C. for 1.5 hours.
e) The blocking buffer was discarded. Each well was washed three times with 200 μL PBST for 3 min each time. A goat anti-mouse enzyme-labeled secondary antibody was diluted 1:5000 by a dilution buffer. The dilution buffer was PBST containing 1% BSA. 100 μL/well of the diluted secondary antibody was added to the ELISA plate. The ELISA plate was incubated at 37° C. for 1 hour.
f) The liquid in the wells was discarded. Each well was washed three times with 200 μL PBST for 5 min each time. Under light-protected conditions, a 3,3′,5,5′-Tetramethylbenzidine (TMB) chromogenic substrate solution was added at 100 μL/well to the ELISA plate. The ELISA plate was incubated at room temperature, protected from light, for 10 minutes. A stop solution (100 μL) was added, and the OD450 nm value was measured.

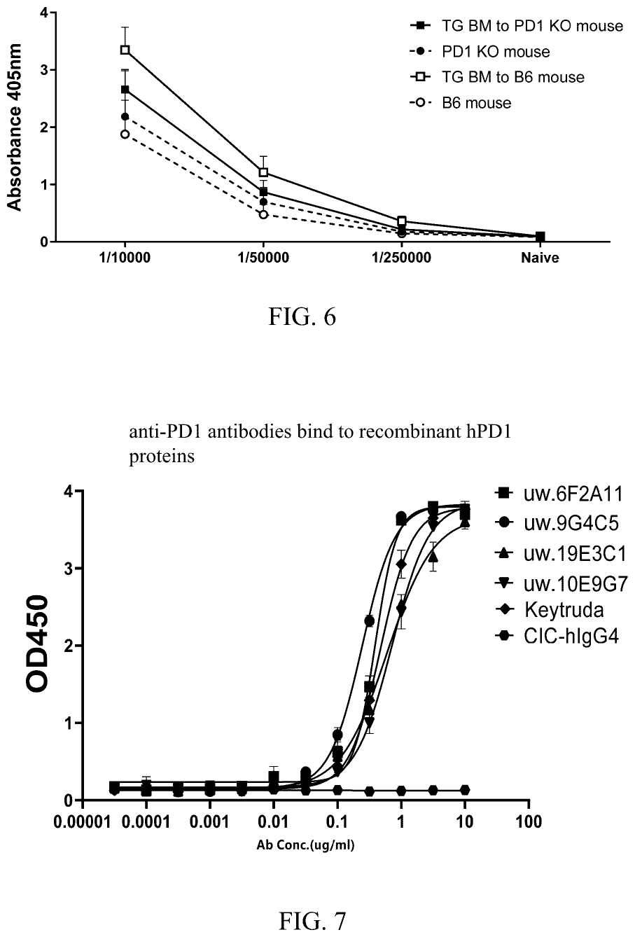

The results show that after diluting the mouse serum 1:250000, the OD405 nm absorbance values of the samples from groups G1-G4 are higher than that of the non-immunized control serum (Naïve). The result indicates that the titer of antigen-specific antibodies in the sera from these four groups reaches 250000. Furthermore, the serum antibody titers in the transplanted and reconstituted recipient mice (G1, G3) are higher than those in the control groups that do not undergo irradiation and transplantation (G2, G4) (shown in FIG. 6).

### Screening and Production of Anti-hPD1 Monoclonal Antibody

### (1) Hybridoma Screening

When the mouse serum titer reached a suitable range, mice from group G1 showing a significant immune response were selected for an intraperitoneal booster immunization using a recombinant hPD1 protein. Each mouse received an intraperitoneal injection of 25 μg of the recombinant hPD1 protein. Three to four days later, the spleens of the mice were harvested. The spleens were ground using a 70 μm sieve and then fused with SP2/0 cells using an electrofusion instrument. The fused cells were plated and cultured in Hypoxanthine-Thymidine (HT) medium for 7 days. The supernatant from the fused hybridoma cells was then assayed by plating with the recombinant hPD1 protein to perform positive ELISA screening for hybridomas.

### (2) Subcloning Screening and Clone Stabilization

The ELISA-positive parental hybridoma clones obtained from fusion were subjected to subcloning. The confirmed positive cells were resuspended and transferred to a 1.5 mL Eppendorf (EP) tube. 1000 μL of HT medium was added. A small number of cells were taken for counting. 100 cells were taken and dissolved in 23 mL of HT medium. 200 μL was added per well for limited dilution in a 1×96-well plate. On day 7 after culturing the subcloned cells, the supernatant from the subcloned cells was assayed by plating with the recombinant hPD1 protein to perform positive ELISA screening for subclones. Concurrently, the supernatant from ELISA-positive subclones was co-incubated with Jurkat-hPD1 cells (purchased from Nanjing Kebai Biotechnology Co., Ltd.), followed by flow cytometry analysis, to screen for binding of the subclone supernatant to the natural hPD1 protein. Finally, four double-positive subclones that bound to both the recombinant hPD1 protein and Jurkat-hPD1 cells were identified and named 6F2A11, 9G4C5, 19E3C1, and 10E9G7. The four positive subclone wells were transferred to 6-well plates for expansion culture. After the cells reached confluence, they were divided into two portions. One portion was cryopreserved in liquid nitrogen for sequencing, and the other portion was cryopreserved as a backup.

### (3) Sequencing of Positive Monoclonal Clones

The screened positive subclone cells (6F2A11, 9G4C5, 19E3C1, and 10E9G7) were lysed. mRNA was extracted and reverse transcribed into cDNA. Using the cDNA as a template, the nucleic acid sequences of the light chain and heavy chain variable regions of the IgG antibody were amplified by PCR, respectively. Sanger sequencing analysis was performed on the heavy chain variable region and the light chain variable region.

TABLE 2
Amino acid sequences of the heavy chain and light chain variable regions, and the
human heavy chain and light chain constant regions of the four positive clones
(6F2A11, 9G4C5, 19E3C1, and 10E9G7)

| Clone | | |
|---|---|---|
| No. | Category | Sequence |
| 6F2A11 | Heavy Chain | QVQLQQSGAELVRPGTSPKISCKASGYIFTYYWLGWVKQRP |
| | Variable Region | GHGLEWIGEIYPGGGYTNYNEKFKGKATLTADTSSSTAYMQ |
| | | LSSLTSEDSAVYFCARFTTVVPFDYWGQGTTLTVSS (SEQ ID |
| | | NO. 1) |
| | Light Chain | DIVMTQSPDSLAVSLGERATMNCKSSQSVLDSSNNKNYLAW |
| | Variable Region | YQQKPGQPPKLLMYWPSTRESGVPDRFRGSGSGTDFTLTISS |
| | | LQAEDVALYYCQQYFYSPWTFGQGTKVEIK (SEQ ID NO. 2) |
| 9G4C5 | Heavy Chain | EVOLVESGGGLVKPGGSLRLSCAVSGFTLSNAWMNWVRQA |
| | Variable Region | PGKGLEWVGRIKTKTDGGTTDYAAPVKGRFTISRDDSKNTL |
| | | YLQMNSLKTEDTAVYYCTTDNWGRDSWGQGTLVTVSS |
| | | (SEQ ID NO. 3) |
| | Light Chain | DIVMTQSPESLAASLGERATINCKSSQSVLYISSNKNYLAWY |
| | Variable Region | QQTPGQPPKLLIYWASTRESGVPDRFSGSGSGTDFTLTISSLQ |
| | | AEDVAVYYCQQYYSSPYTFGQGTKLEIK (SEQ ID NO. 4) |
| 19E3C1 | Heavy Chain | QVQLQQSGAELVRPGTSVKISCKASGYTFTNYWLGWVKQR |
| | Variable Region | PGHGLEWIGDIYPGGGYTNYNEKFKVKATLTADTSSSTAYM |
| | | QLSSLTSEDSAVYFCARFTTVVPFDYWGQGTTLTVSS (SEQ |
| | | ID NO. 5) |
| | Light Chain | DIVMTQSPDSLAVSLGERATINCKSSQSVLYSSNNKNYFAWY |
| | Variable Region | QQKPGQPPNLLINWASTRESGVPDRFTGSGSGTDFTLTISSLQ |
| | | AEDVAVYFCQQYYSPPWSFGQGTKVEIK (SEQ ID NO. 6) |
| 10E9G | Heavy Chain | QVQLQQSGAELVRPGTSLKISCKASGYTFINYWLGWVKQRP |
| | Variable Region | GHGLEWIGDIYPGGGYTNYNEKFKGKATLTADTSSSIAHLQ |
| | | LSSLTSEDSAVYFCARFTTVVPFDYWGQGTTLTVSS (SEQ ID |
| | | NO. 7) |
| | Light Chain | DIVMTQSPDSLAVSLGERATINCKSSQSVLYSSNNKHYLAW |
| | Variable Region | YQQKPGQPPKLLIYWPSTREIGVPDRFRGSGSGTDFTLTISSL |
| | | QAEDVALYYCQQYYSTPWTFGQGTKVEIK (SEQ ID NO. 8) |
| | IgG1 Heavy Chain | ASTKGPSVFPLAPSSKSTSGGTAALGCLVKDYFPEPVTVSWN |
| | Constant Region | SGALTSGVHTFPAVLQSSGLYSLSSVVTVPSSSLGTQTYICNV |
| | | NHKPSNTKVDKKVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSRDELTKNQVSLTC |
| | | LVKGFYPSDIAVEWESNGQPENNYKTTPPVLDSDGSFFLYSK |
| | | LTVDKSRWQQGNVFSCSVMHEALHNHYTQKSLSLSPGK |
| | | (SEQ ID NO. 9) |
| | IgG1 Light Chain | RTVAAPSVFIFPPSDEQLKSGTASVVCLLNNFYPREAKVQWK |
| | Constant Region | VDNALQSGNSQESVTEQDSKDSTYSLSSTLTLSKADYEKHK |
| | | VYACEVTHQGLSSPVTKSFNRGEC (SEQ ID NO. 10) |

### (4) Production of Anti-hPD1 Antibody

Using 6F2A11 as an example, a heavy chain antibody sequence of 6F2A11 (a heavy chain variable region from 6F2A11 positive clone sequencing and a constant region of human IgG1) and a light chain antibody sequence of 6F2A11 (a light chain variable region from 6F2A11 positive clone sequencing and a constant region of human Kappa chain) were synthesized by gene synthesis. Corresponding nucleic acid sequences were designed based on the synthesized heavy chain antibody sequence and light chain antibody sequence of 6F2A11, respectively. The nucleic acid sequences were ligated to a PTT5 vector, respectively, to obtain a 6F2A11 heavy chain PTT5 vector and a 6F2A11 light chain PTT5 vector. The two vectors were transiently transfected into 293T cells simultaneously. A supernatant protein of the 293T cells was harvested. The Anti-hPD1 antibody was obtained by Protein A affinity chromatography purification and named as uw.6F2A11.

Anti-hPD1 antibodies uw.9G4C5, uw. 19E3C1, and uw. 10E9G7 of positive clones 9G4C5, 19E3C1, and 10E9G7 were produced by referring to the above manner.

### In Vitro Activity Evaluation of Anti-hPD1 Antibody

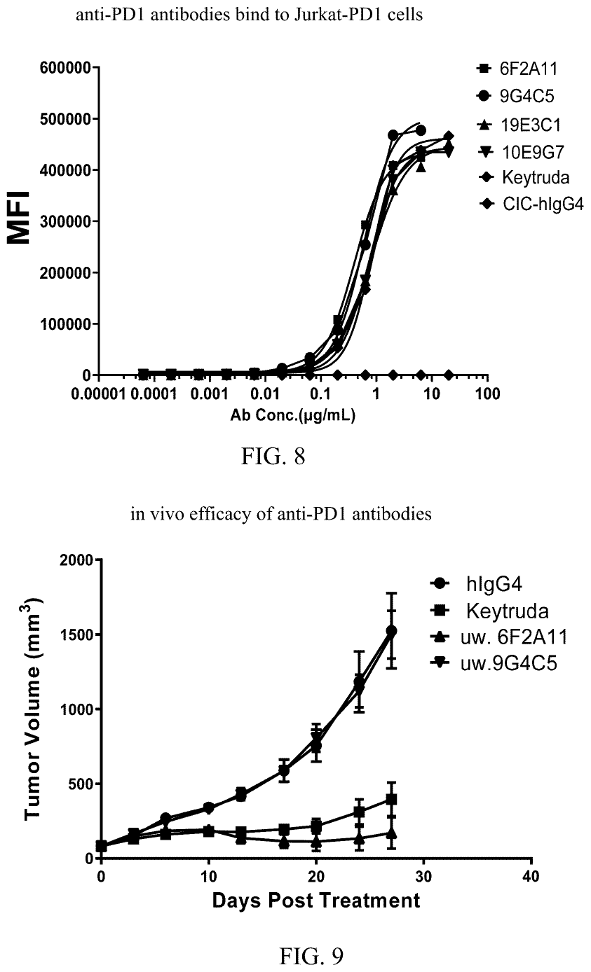

Four antibodies (uw.6F2A11, uw.9G4C5, uw. 19E3C1, and uw. 10E9G7) obtained by the above purification were selected for in vitro activity evaluation. First, a recombinant hPD1 protein was plated. The four antibodies at different gradients were added, respectively. A binding effect of the antibodies to the recombinant hPD1 protein was detected. Results are shown in FIG. 7. The results show that uw.6F2A11, uw.9G4C5, uw. 19E3C1, and uw. 10E9G7 all bind to the recombinant hPD1 protein, and binding of uw.6F2A11 and uw.9G4C5 to the recombinant hPD1 protein is better than that of a positive control keytruda. Further, the purified antibodies were co-incubated with Jurkat-hPD1 cells. Then, an anti-IgG1-Fc secondary antibody conjugated with APC was added. A binding effect of the purified antibodies to the Jurkat-hPD1 cells was evaluated by an APC mean fluorescence intensity (MFI) of the secondary antibody. Results are shown in FIG. 8. The results show that uw.6F2A11, uw.9G4C5, uw. 19E3C1, and uw. 10E9G7 all bind to the Jurkat-hPD1 cells. hIgG4 was used as a negative control, and Keytruda was used as a positive control.

TABLE 3
Affinity data of anti-hPD1 antibody

| | Capture | Analyte 1 | | Koff | Kon | KD |
|---|---|---|---|---|---|---|
| Ligand | Conc. | Solution | FullR2 | (1/s) | (1/Ms) | (M) |
| Keytruda | 1 μg/mL | Human PD1 | 0.991 | 2.11E−003 | 7.42E+005 | 2.84E−009 |
| 6F2A11 | 1 μg/mL | Human PD1 | 0.996 | 1.18E−003 | 4.79E+005 | 2.46E−009 |
| 9G4C5 | 1 μg/mL | Human PD1 | 0.988 | 4.77E−004 | 1.00E+005 | 4.76E−009 |
| 19E3C1 | 1 μg/mL | Human PD1 | 0.954 | 7.35E−003 | 3.54E+005 | 2.08E−008 |
| 10E9G7 | 1 μg/mL | Human PD1 | 0.984 | 1.81E−003 | 2.87E+005 | 6.32E−009 |

### In Vivo Efficacy Evaluation of Anti-hPD1 Monoclonal Antibody

Using two antibodies, uw.9G4C5 and uw.6F2A11, as examples, in vivo efficacy activity evaluation was further performed. An in vivo efficacy experiment was performed based on a B6-hPD1 mouse subcutaneous inoculation MC38 model. Mouse colon cancer cells MC38 at a logarithmic growth phase were subcutaneously inoculated into 6-8-week-old B6-hPD1 mice (products of GemPharmatech Co., Ltd., Jiangsu, China). On day 6 after inoculation, when an average tumor volume reached 82.13 mm³, 32 mice were selected and randomly grouped into an hIgG4 (negative control) group, a Keytruda (positive control) group, a uw.9G4C5 group, and a uw.6F2A11 group (n=8) according to tumor volumes. The mice were treated with corresponding drugs (hIgG4, Keytruda, uw.9G4C5, and uw.6F2A11), respectively. Specific administration groups are shown in Table 4 below. Administration was performed twice a week for a total of 6 administrations. Results are shown in FIG. 9. The results show that the antibody uw.9G4C5 has a significant tumor growth inhibition effect. The tumor growth inhibition effect of the antibody uw.9G4C5 is better than that of the positive drug Keytruda.

TABLE 4
Administration groups for in vivo efficacy of anti-PD1 antibody

| | | Adminis- | | Adminis- |
|---|---|---|---|---|
| | Count of | tration | Adminis- | tration |
| | Animals | Dose | tration | Frequency |
| Group | (n) | (mg/kg) | Route | and Period |
| hIgG4 | 6 | 5 | i.p. | BIW × 3 week |
| Keytruda | 6 | 5 | i.p. | BIW × 3 week |
| uw.6F2A11 | 6 | 5 | i.p. | BIW × 3 week |
| uw.9G4C5 | 6 | 5 | i.p. | BIW × 3 week |

### Example 3 Construction of Immune-Reconstituted Mouse Via Fetal Liver Cell Transplantation

To test whether stem cells derived from mouse embryos can reconstruct an immune system in a transplant recipient, in the present example, fetal liver cells of a suitable gestational age were isolated and transplanted into a recipient mouse subjected to immunosuppressive pretreatment (Table 5). Specific operations were as follows.

Immunosuppressive pretreatment of the recipient mouse: 4-week-old B6 recipient mice were selected for irradiation pretreatment. The mouse was fed with an antibiotic for 7 days before irradiation. The mouse was continuously fed with the antibiotic for 14 days after irradiation (Table 5). A donor embryo was an F1 generation heterozygous embryo (129×BALB/c F1 fetal liver) obtained by mating BALB/cJGpt (a product of GemPharmatech Co., Ltd., Jiangsu, China, a strain number of N000020) with 129S1/SvImJGpt (a product of GemPharmatech Co., Ltd., Jiangsu, China, a strain number of N000017). The recipient mouse was a B6 mouse (a product of GemPharmatech Co., Ltd., Jiangsu, China, a strain number of N000013).

Extraction of fetal liver cells of a donor mouse: a suitable pregnant mouse was euthanized. A uterus was dissected, an embryo was taken out, and a fetal membrane was peeled off. The embryo was repeatedly washed with 4° C. PBS. An anterior abdominal wall of the embryo was finely separated. A fetal liver was completely taken out and placed in a sterile culture dish containing 4° C. PBS for washing. After rinsing three times, the fetal liver was coarsely cut. The fetal liver was ground with ground glass and filtered through a 40 μm filter. The fetal liver was carefully aspirated into a 50 mL centrifuge tube with a sterile pipette. Cells were collected for transplantation after RBC Lysis Buffer lysed red blood cells.

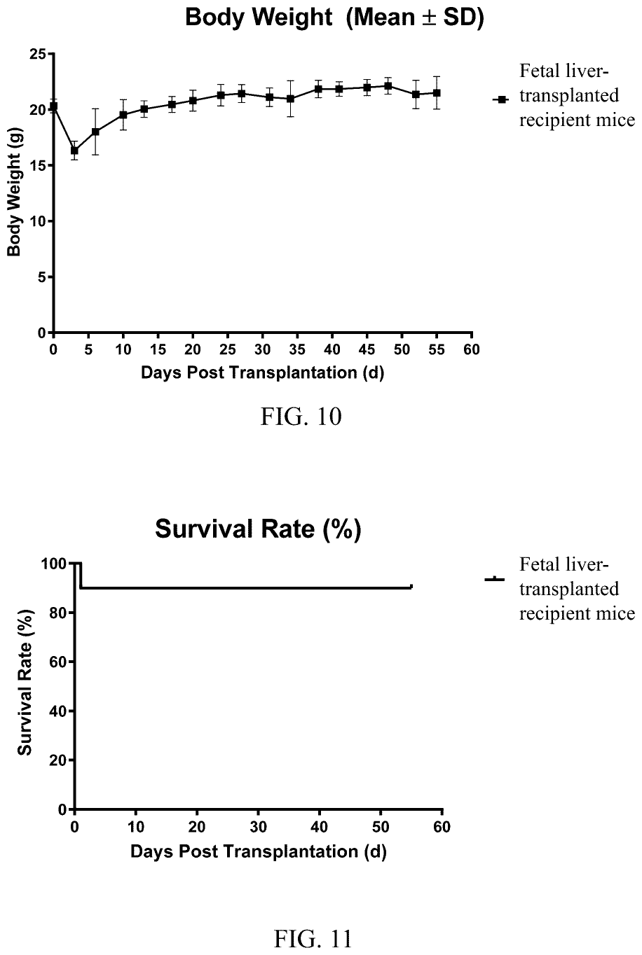

Fetal liver cell transplantation: after irradiation, the fetal liver cells were transplanted into irradiated recipient mice via tail vein injection. The day of irradiation was defined as DO. After transplantation, weekly observations and weighing were performed to record body weight and survival rate. The results are shown in FIG. 10 and FIG. 11.

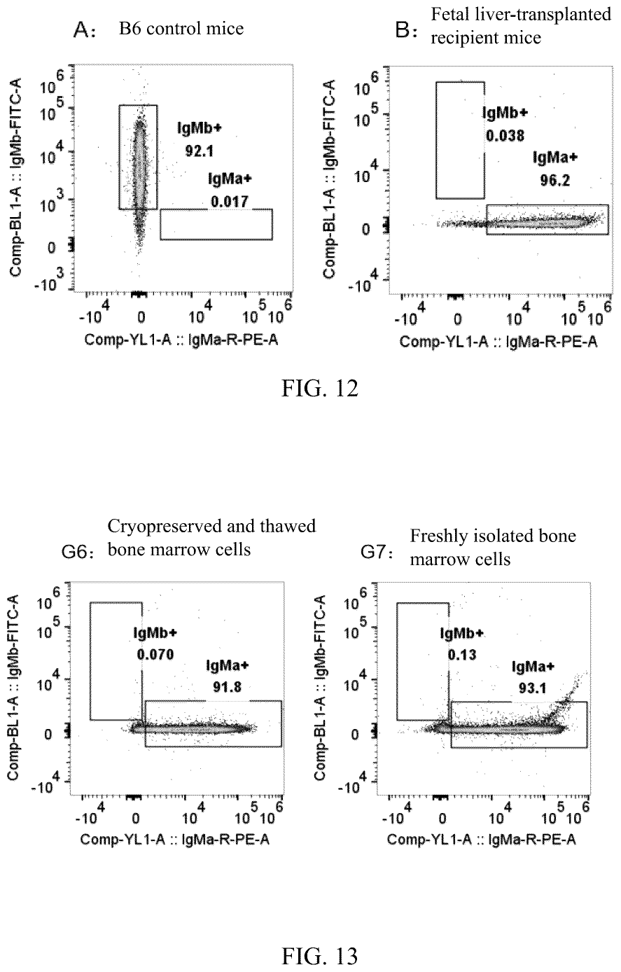

The results show that mouse fetal liver cells can reconstitute the immune system in recipient mice. After transplantation, the body weight of the recipient mice increases steadily (FIG. 10), and the survival rate of the fetal liver transplantation group is 90% (FIG. 11). FIG. 12 is a diagram showing results of the B cell (mCD19+) origin analysis for the mice reconstituted by the fetal liver cells transplantation (also referred to as fetal liver-transplanted recipient mice). In FIG. 12, A represents the B cell origin analysis of B6 control mice that do not receive fetal liver cell transplantation, and B represents the B cell origin analysis of the fetal liver recipient mice. Since B6 background mice have IgM of b allotype, while 129 and BALB/c background mice have IgM of an allotype, flow cytometry was used to detect the different IgM allotypes for distinction. mIgM-A is the protein expressed on the surface of B cells from 129×BALB/c background mice, and mIgM-B is the protein expressed on the surface of B cells from the B6 mice. The results show that almost all B cells (mCD19+) are derived from the donor mice, demonstrating that fetal liver transplantation can reconstitute the immune system in recipient mice. In this example, F1 hybrid mice from a cross between 129 and BALB/c strains were used as stem cell donors, which were transplanted into the B6 recipient mice. The results show that the transplantation and reconstitution method proposed in the present disclosure can be performed between different genetic backgrounds.

TABLE 5
Experiment groups for fetal liver cell
transplantation reconstituted animals

| | Count | Recipient | Donor Cell |
|---|---|---|---|
| Group | (n) | Mouse | Source |
| G5 | 10 | B6 | 129 × BALB/c |
| | | | F1 fetal liver |

Note:
G represents a group; n represents a count of animals.

### Example 4 Construction of Immune-Reconstituted Mice Using Cryopreserved and Thawed Stem Cells

In this example, bone marrow cells cryopreserved in liquid nitrogen were thawed and transplanted into recipient mice that had undergone immunosuppressive pretreatment (referring to the manner in Example 1, specific groups are shown in Table 6) for immune reconstitution. Origin analysis was performed by the flow cytometry. The results, as shown in FIG. 13, show that cryopreserved and thawed bone marrow cells can reconstitute the immune system in recipient mice.

TABLE 6
Cryopreserved and thawed bone marrow cell transplantation
and reconstitution experiment

| | | | Count of | |
|---|---|---|---|---|
| | Count | Recipient | Transplanted | Donor Cell |
| Group | (n) | Mouse | Cells | Source |
| G6 | 10 | B6 | 1 × 10⁷ | Cryopreserved and Thawed |
| | | | cells/mouse | Bone Marrow Cells |
| G7 | 10 | B6 | 1 × 10⁷ | Freshly Isolated Bone |
| | | | cells/mouse | Marrow Cells |

### Example 5 Immunization and Human Antibody Screening Experiment for Different Targets

Bone marrow cells from donor mice carrying human immunoglobulin variable region-encoding genes were obtained according to the manner in Example 2 and transplanted into corresponding target antigen-encoding gene knockout recipient mice that had undergone immunosuppressive pretreatment (referring to the manner in Example 1, specific groups are shown in Table 7) for immune reconstitution. Successfully reconstituted mice were immunized to screen for antibodies encoded by human genes. The target antigen-encoding gene knockout recipient mice were TIGIT KO mice (products of GemPharmatech Co., Ltd., Jiangsu, a strain number of T037162) and LAG3 KO mice (products of GemPharmatech Co., Ltd., Jiangsu, a strain number of T002755). Origin analysis of immune cells and measurement of immune serum titers were performed after bone marrow transplantation in TIGIT KO mice and LAG3 KO mice.

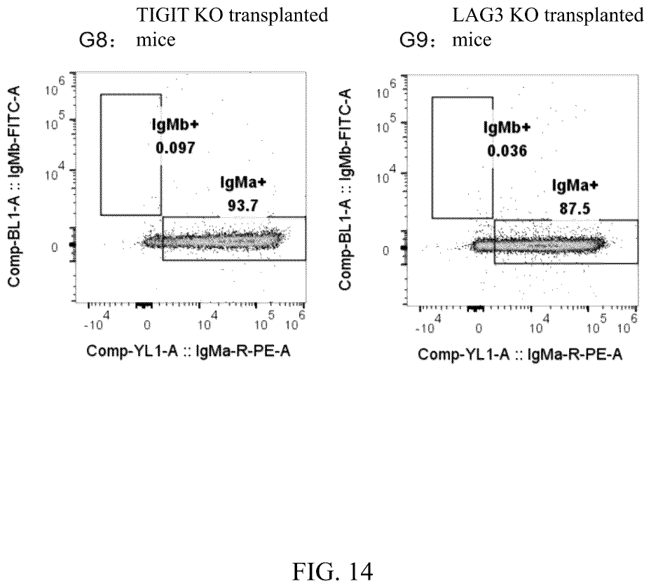

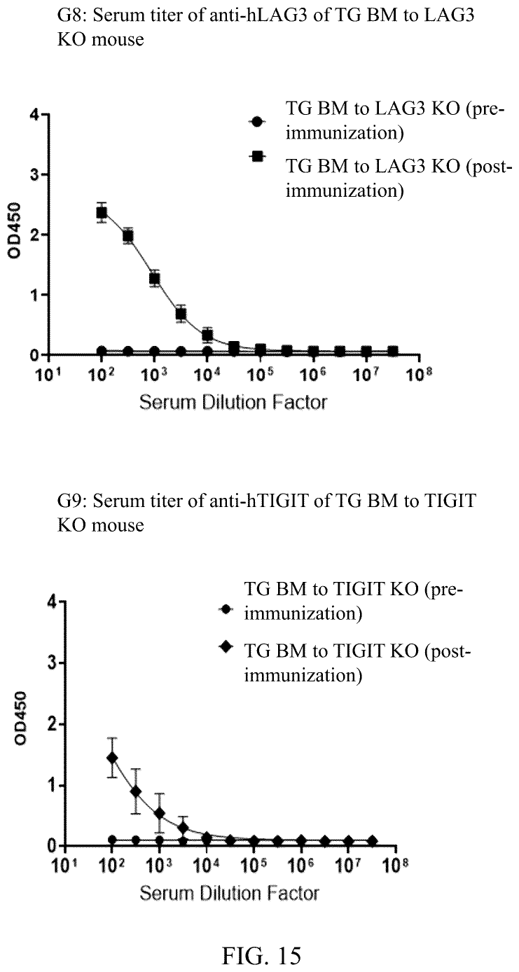

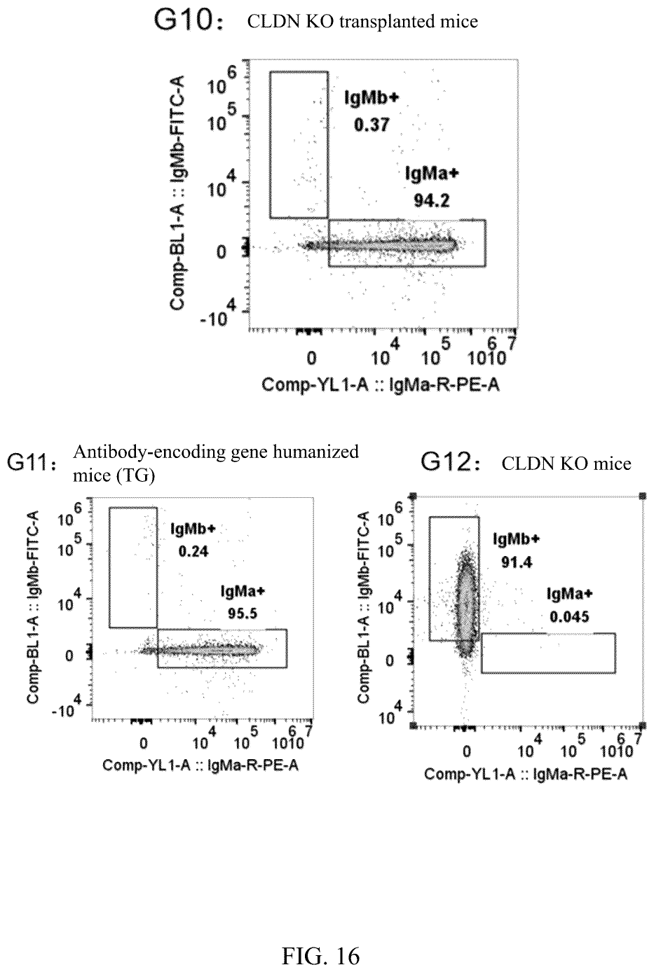

The results, as shown in FIGS. 14 and 15, demonstrate that bone marrow cells from antibody-encoding gene humanized mice can successfully reconstitute the immune system in various gene knockout mice. After immunization with recombinant hTIGIT protein antigen, the transplanted and reconstituted TIGIT KO mice generate an immune response and produce specific antibodies with titers exceeding 100,000. After immunization with recombinant hLAG3 protein antigen, the transplanted and reconstituted LAG3 KO mice generate an immune response and produce specific antibodies with titers exceeding 100,000. The results demonstrate that the technical solution described in the present disclosure is applicable to human antibody screening for different targets.

TABLE 7
Screening experiment of human antibodies for different targets

| | | | Count of | Source of |
|---|---|---|---|---|
| | Count | Recipient | Transplanted | Donor Bone |
| Group | (n) | Mouse | Cells | Marrow Cells |
| G8 | 5 | TIGIT | 1 × 10⁷ | Antibody-Encoding |
| | | KO Mouse | cells/mouse | Gene Humanized Mouse |
| G9 | 5 | LAG3 | 1 × 10⁷ | Antibody-Encoding |
| | | KO Mouse | cells/mouse | Gene Humanized Mouse |

### Example 6 Breaking B Cell Immune Tolerance and Enhancing Immune Response Through Bone Marrow Transplantation and Reconstitution

Bone marrow cells from donor mice carrying human immunoglobulin variable region encoding genes were obtained according to the manner in Example 2. The bone marrow cells were transplanted into corresponding target antigen-encoding gene knockout recipient mice that had undergone immunosuppressive pretreatment (according to the manner in Example 1) for immune reconstitution. According to Table 8, successfully reconstituted mice, antibody-encoding gene humanized mice (TG), and CLDN KO mice were immunized to evaluate whether B cell peripheral immune tolerance was broken, and immune response was enhanced after bone marrow transplantation. The target antigen-encoding gene knockout recipient mice were CLDN KO mice (products of Jiangsu GemPharmatech Co., Ltd., a strain number of T014374).

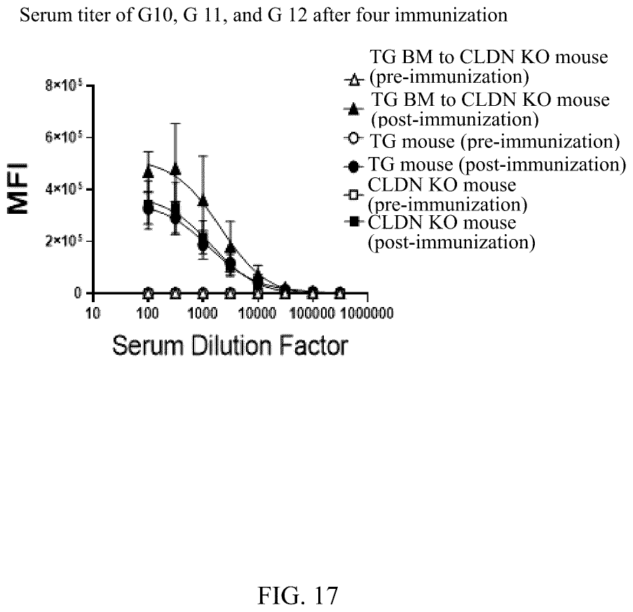

Results, as shown in FIGS. 16 and 17, indicate that bone marrow cells from antibody-encoding gene humanized mice can successfully reconstitute the immune system in different gene knockout mice. After immunization with HEK293-hClaudin18.2 cells (commercially available), the transplanted and reconstituted CLDN KO mice can generate an immune response. The immune response is higher than that in antibody-encoding gene humanized mice (TG) and CLDN KO mice, which indicates that the immune response is enhanced after bone marrow transplantation from antibody-encoding gene humanized mice (TG) to CLDN KO mice.

TABLE 8
Experimental design and grouping for immune tolerance

| | Count | Donor | Recipient | Donor | | Immunization |
|---|---|---|---|---|---|---|
| Group | (n) | Mouse | Mouse | Cells | Immunogen | Frequency |
| G10 | 3 | Antibody- | CLDN | Bone | HEK293T- | Administered |
| | | Encoding | Gene | Marrow | hClaudin18.2 | once every |
| | | Gene | Knockout | Cells | | 2 weeks, a |
| | | Humanized | Mouse | | | total of |
| | | Mouse (TG) | (CLDN KO) | | | 4 doses |
| G11 | 3 | Antibody- | None | / | HEK293T- | Administered |
| | | Encoding | | | hClaudin18.2 | once every |
| | | Gene | | | | 2 weeks, a |
| | | Humanized | | | | total of |
| | | Mouse (TG) | | | | 4 doses |
| G12 | 3 | None | CLDN | None | HEK293T- | Administered |
| | | | Gene | | hClaudin18.2 | once every |
| | | | Knockout | | | 2 weeks, a |
| | | | Mouse | | | total of |
| | | | (CLDN KO) | | | 4 doses |

What is claimed is:

1. A method for producing an antibody, comprising:
immunizing a recipient animal with an antigen and obtaining an antigen-specific antibody, wherein the recipient animal is an animal transplanted with stem cells with differentiation potential by immune system reconstitution, the stem cells with differentiation potential are derived from a donor animal carrying one or more human immunoglobulin variable region gene segments, and the recipient animal is a non-human mammal.

2. The method according to claim 1, wherein the stem cells with differentiation potential comprise at least one of bone marrow cells, fetal liver cells, hematopoietic stem cells, pluripotent stem cells, or induced pluripotent stem cells (iPSCs).

3. The method according to claim 2, wherein the stem cells with differentiation potential are freshly isolated cells or cryopreserved and thawed cells.

4. The method according to claim 1, wherein the immune system reconstitution comprises: inducing immunodeficiency or suppression in an immune system of the recipient animal by at least one of myeloablation, immunosuppression and immunomodulation using a low-toxicity drug, or genetic modification; and transplanting the stem cells with differentiation potential into the recipient animal.

5. The method according to claim 4, wherein the myeloablation comprises eliminating hematopoietic stem cells in a bone marrow of the recipient animal using a sub-lethal dose of ionizing radiation or chemical means; the low-toxicity drug is a differentiation signaling blocker or a drug that inhibits stem cell proliferation; and the induced immunodeficiency or suppression in the immune system of the recipient animal by the genetic modification includes developmental or differentiation defects of the hematopoietic stem cells or developmental defects of a plurality of immune cells.

6. The method according to claim 5, wherein the ionizing radiation is X-ray irradiation or Co-60 gamma-ray irradiation, and the chemical means is administering at least one of busulfan, cyclophosphamide, or melphalan to the recipient animal.

7. The method according to claim 1, wherein the recipient animal is a rodent.

8. The method according to claim 1, wherein the recipient animal is selected from a group consisting of a rat, a mouse, a rabbit, a sheep, a camelid, and a non-human primate.

9. The method according to claim 1, wherein the recipient animal is a wild-type animal or an animal carrying a non-natural endogenous gene.

10. The method according to claim 9, wherein the animal carrying the non-natural endogenous gene is a target antigen-encoding gene knockout animal.

11. The method according to claim 1, comprising:
(a) subjecting the recipient animal to immunosuppressive pretreatment to obtain an immunosuppressed recipient animal; providing the donor animal carrying one or more human immunoglobulin variable region gene segments, and collecting the stem cells with differentiation potential from the donor animal and transplanting the stem cells into the immunosuppressed recipient animal to obtain an immune-reconstituted recipient animal with immune cells derived from the donor animal;
(b) immunizing the immune-reconstituted recipient animal obtained in step (a) with the antigen; and
(c) confirming that the immunized immune-reconstituted recipient animal is an animal capable of producing an antibody binding to the antigen, collecting immune cells of the recipient animal, and further producing a human antibody by using the collected immune cells.

12. The method according to claim 11, wherein the immunosuppressive pretreatment comprises eliminating hematopoietic stem cells in a bone marrow of the recipient animal using a sub-lethal dose of ionizing radiation.

13. A human antibody, which is produced by the method according to claim 1.

14. A pharmaceutical composition, comprising the human antibody of claim 13.

15. An immune-reconstituted mouse, which is transplanted with stem cells with differentiation potential, wherein the stem cells with differentiation potential are derived from a transgenic mouse carrying one or more human immunoglobulin variable region gene segments.

16. The immune-reconstituted mouse according to claim 15, wherein the stem cells with differentiation potential comprise at least one of bone marrow cells, fetal liver cells, hematopoietic stem cells, pluripotent stem cells, or induced pluripotent stem cells (iPSCs).

17. The immune-reconstituted mouse according to claim 15, wherein the immune-reconstituted mouse is a target antigen-encoding gene knockout mouse capable of producing a human antibody against the target antigen after antigen immunization.

18. The immune-reconstituted mouse according to claim 15, which is obtained by inducing immunodeficiency or suppression in an immune system of a mouse by at least one of myeloablation, immunosuppression and immunomodulation using a low-toxicity drug, and genetic modification; and transplanting the stem cells with differentiation potential into the mouse.

19. The immune-reconstituted mouse according to claim 15, wherein human immunoglobulin is selected from a group consisting of IgG, IgM, IgA, IgD, and IgE.

20. The immune-reconstituted mouse according to claim 15, wherein a variable region is at least one of regions encoded by gene segments formed by human V_{H}D_{H}J_{H} rearrangement, human V_{K}J_{K} rearrangement, or human V_{L}J_{L} rearrangement.


SEQUENCE LISTING (10 sequences)

SEQ ID NO: 1 - protein, 118 aa, synthetic construct
QVQLQQSGAELVRPGTSPKISCKASGYIFTYYWLGWVKQRPGHGLEWIGEIYPGGGYTNYNEKFKGKATLTADTSSSTAYMQLSSLTSEDSAVYFCARFTTVVPFDYWGQGTTLTVSS

SEQ ID NO: 2 - protein, 113 aa, synthetic construct
DIVMTQSPDSLAVSLGERATMNCKSSQSVLDSSNNKNYLAWYQQKPGQPPKLLMYWPSTRESGVPDRFRGSGSGTDFTLTISSLQAEDVALYYCQQYFYSPWTFGQGTKVEIK

SEQ ID NO: 3 - protein, 118 aa, synthetic construct
EVQLVESGGGLVKPGGSLRLSCAVSGFTLSNAWMNWVRQAPGKGLEWVGRIKTKTDGGTTDYAAPVKGRFTISRDDSKNTLYLQMNSLKTEDTAVYYCTTDNWGRDSWGQGTLVTVSS

SEQ ID NO: 4 - protein, 113 aa, synthetic construct
DIVMTQSPESLAASLGERATINCKSSQSVLYISSNKNYLAWYQQTPGQPPKLLIYWASTRESGVPDRFSGSGSGTDFTLTISSLQAEDVAVYYCQQYYSSPYTFGQGTKLEIK

SEQ ID NO: 5 - protein, 118 aa, synthetic construct
QVQLQQSGAELVRPGTSVKISCKASGYTFTNYWLGWVKQRPGHGLEWIGDIYPGGGYTNYNEKFKVKATLTADTSSSTAYMQLSSLTSEDSAVYFCARFTTVVPFDYWGQGTTLTVSS

SEQ ID NO: 6 - protein, 113 aa, synthetic construct
DIVMTQSPDSLAVSLGERATINCKSSQSVLYSSNNKNYFAWYQQKPGQPPNLLINWASTRESGVPDRFTGSGSGTDFTLTISSLQAEDVAVYFCQQYYSPPWSFGQGTKVEIK

SEQ ID NO: 7 - protein, 118 aa, synthetic construct
QVQLQQSGAELVRPGTSLKISCKASGYTFINYWLGWVKQRPGHGLEWIGDIYPGGGYTNYNEKFKGKATLTADTSSSIAHLQLSSLTSEDSAVYFCARFTTVVPFDYWGQGTTLTVSS

SEQ ID NO: 8 - protein, 113 aa, synthetic construct
DIVMTQSPDSLAVSLGERATINCKSSQSVLYSSNNKHYLAWYQQKPGQPPKLLIYWPSTREIGVPDRFRGSGSGTDFTLTISSLQAEDVALYYCQQYYSTPWTFGQGTKVEIK

SEQ ID NO: 9 - protein, 330 aa, synthetic construct [sequence omitted]

SEQ ID NO: 10 - protein, 107 aa, synthetic construct [sequence omitted]